(19) United States
(12) Patent Application Publication
(10) Pub. No.: US 2006/0121020 A1
(43) Pub. Date: Jun. 8, 2006

(54) Mixture preparation for enhancing absorption of immunoglobulins in postnatal domestic mammals, and the method thereof

(75) Inventors: Hachiro Kamada, Sapporo-shi (JP); Itoko Nonaka, Tsukuba-shi (JP)

Correspondence Address: PITNEY HARDIN LLP, 7 TIMES SQUARE, NEW YORK, NY, 10036-7311, US

(73) Assignee: Incorporated Administrative Agency, National Agriculture and Bio-oriented Research Organizaion

(21) Appl. No.: 11/195,455

(22) Filed: Aug. 2, 2005

(30) Foreign Application Priority Data
Dec. 7, 2004 (JP) JP2004-354190

(51) Int. Cl. A61K 39/395; A61K 35/20; A61K 33/04

(52) U.S. Cl. 424/130.1; 424/702; 424/535

(57) ABSTRACT

Disclosed is a mixture preparation containing an addition of selenium for enhancing absorption of immunoglobulins administered to postnatal domestic mammals (e.g., bovine), and the method thereof. As an example of a selenium compound added, sodium selenite is particularly effective. Further, the mixture preparation may contain raw milk, colostrum and/or cheese whey as immunoglobulin sources.

### BACKGROUND OF THE INVENTION

1. Field of the Invention

The present invention relates to a mixture preparation containing selenium for enhancing absorption of immunoglobulins administered to postnatal domestic mammals (e.g., bovine), and the method thereof.

2. Description of the Related Art

An immunoglobulin is a protein, which has a role in eliminating pathogenic microbes and is essential to mammals. In particular, postnatal calves cannot produce immunoglobulins on their own and cannot protect themselves from infectious diseases without intaking immunoglobulins from the outside sources. Since the first milking colostrum secreted post-parturition by maternal cows contains a high concentration of immunoglobulins, calves normally acquire immunoglobulins by ingestion of such colostrum. However, the absorption capacity of immunoglobulins in the small intestines of calves decreases as a function of time post-parturition and almost comes to a nil within 24 hours post-parturition. Because of this phenomenon, the absorption of immunoglobulins is normally accomplished by early administration of colostrum to postnatal calves. In this regard, the important points in administering colostrum are as follows: (1) the timing of administering colostrum, (2) the administration amount of colostrum, and (3) the quality of colostrum. As to these conditions, they are described in detail in “Management of Colostrum,” Journal of Clinical Veterinary Medicine, Vol. 19, No. 3 (2001), pp. 18-38. To rephrase, it is critical to administer colostrum as early as possible in a sufficient amount under clean and sanitary conditions.

However, there are many cases in which the above supply conditions cannot be met on account of various reasons. For example, there may be cases of an insufficient amount of colostrum for primiparous cows or an insufficient amount of immunoglobulins in colostrum. The absorption capacity of immunoglobulins in calves may change due to stress or temperature. For these reasons, it cannot be said that the amount of absorption of immunoglobulins in postnatal domestic mammals is sufficient under these circumstances, and the rate of occurrences of infectious diseases in immature calves and the attrition rate have not come down to the satisfactory levels. Accordingly, in order to further enhance resistance of calves against infectious diseases, there remains a need for enhancing absorption capacity of immunoglobulins in postnatal domestic mammals such as calves.

### SUMMARY OF THE INVENTION

The relations between the constituents contained in colostrum and the absorption capacity of immunoglobulins in calves have not yet been clarified. The roles of minerals in mammals are gradually been elucidated, and the important roles of certain types of minerals in body have been shown. It is known that for example selenium possesses anti-cancerous activities and activates thyroid hormones. The inventors herein have been looking at the roles of selenium in bovine and have found that the enhancement of absorption capacity of immunoglobulins is accomplished in the intestines of calves by adding selenium to colostrum. In this regard, the absorption of immunoglobulins at the intestinal tracts occurs by pinocytosis of epithelial cells of the intestinal tracts. Selenium is thought to somehow facilitate this pinocytosis process.

Accordingly, the present invention provides a mixture preparation containing an addition of a selenium compound, and a method of enhancing absorption of immunoglobulins in the intestines of postnatal domestic mammals by way of ingestion of immunoglobulins in conjunction with selenium, thereby improving resistance to infectious diseases in postnatal domestic mammals and reducing the attrition rate thereof.

### BRIEF DESCRIPTION OF THE DRAWINGS

FIG. 1 is a schematic diagram of one of the embodiments of the present invention.

FIG. 2 is a comparative graph, which shows the IgG concentrations in plasma of calves administered with colostrum with selenium, in which 100 represents the IgG concentration in plasma of calves administered with colostrum without selenium. To calves administered with colostrum with selenium, selenium was added to the colostrum feeds respectively up to the fourth feeds.

FIG. 3 is a comparative graph, which shows the IgG concentrations in plasma of calves administered with colostrum with selenium, in which 100 represents the IgG concentration in plasma of calves administered with colostrum without selenium. To calves administered with colostrum with selenium, selenium was added only to the first colostrum feeds.

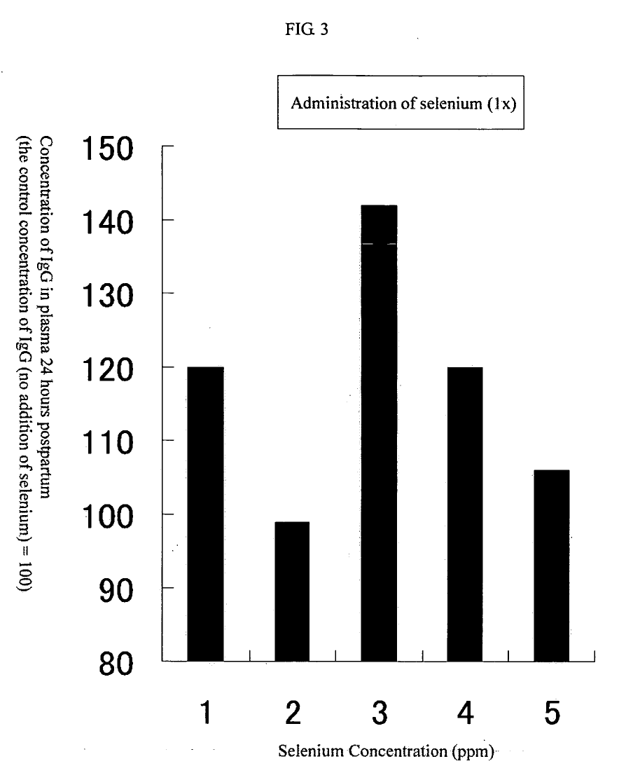

FIG. 4 is a comparative graph, which shows the selenium concentrations in plasma of calves fed with colostrum with or without selenium.

### DETAILED DESCRIPTION OF THE PREFERRED EMBODIMENTS

Hereinafter, the embodiments of the present invention will be described in particular details.

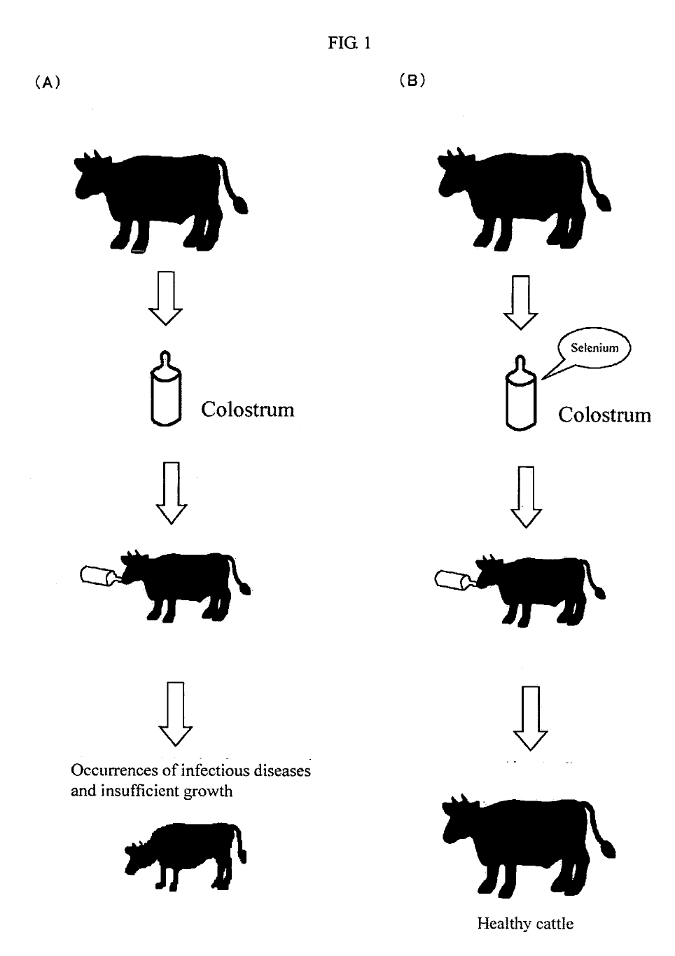

FIG. 1 is a schematic diagram of one of the embodiments of the present invention. As an illustration in FIG. 1(a), where normal colostrum is administered to calves, if the intake thereof is not sufficient, infectious diseases and insufficient growth are more likely before maturity, leading to a higher death rate of calves in general. Meanwhile, as in the present invention, where colostrum with selenium is administered to calves, the absorption of immunoglobulins in calves is enhanced, leading to a higher rate of healthy calves in maturity.

One of the embodiments of the present invention is a mixture preparation for enhancing absorption of immunoglobulins in postnatal domestic mammals (e.g., calves), which comprises an addition of a selenium compound so that the concentration of selenium is 0.2 to 5 ppm after such addition. Immunoglobulins refer to proteins, which are synthesized and secreted by B lymphocytes, and which possess the functions of identifying and eliminating pathogenic microbes. Examples of immunoglobulins include A, G, and M types. Immunoglobulins A (IgA), which exist in salvia, digestive fluid, and phlegm, play an important role in the defense system of mucous membranes. Immunoglobulins G (IgG), which exist in blood, possess for example the functions of fighting against invading microbes or foreign substances in body, and activating protein complements. Immunoglobulins M (IgM), which appear at the outset of antigenic stimulation, also have the functions of fighting against microbes or foreign substances, and activating protein complements. Here, “domestic mammals” include all forms of life acquiring immunoglobulins by postpartum feeding from maternal sources, e.g., human, bovine, porcine, and equine.

Selenium is essential to mammals, and the deficiencies of selenium result in a variety of pathological symptoms. On the other hand, selenium can be toxic as well, and an excessive consumption may result in other forms of pathological symptoms. Accordingly, the selenium concentration in the mixture preparation is at 0.2 to 5 ppm, or preferably 2.5 to 3.5 ppm. If the selenium concentration is less than the above lower limit, the absorption of immunoglobulins is insufficient, and if the mineral content is more than the above upper limit, there may be harmful effects on mammals due to excessive selenium.

Selenium compound(s) added to the mixture preparation can be one or more compounds selected from a group consisting of sodium selenite, sodium selenate, potassium selenite, and potassium selenate. Selenium is essential in the synthesis of enzymes (glutathione peroxidase), which detoxify activated oxygen, and is an important antioxidant nutrient for life support. In particular, selenium is known to have the functions of preventing cancer and regulating thyroid hormones. In regard to the absorption of immunoglobulins at the intestinal tracts, selenium is thought to somehow facilitate the pinocytosis process occurring at the intestinal tracts by way of epithelial cells.

Other examples of compounds containing selenium include compounds of selenium with an oxidation number of −2, such as Na₂Se, CaSe, ZnSe, CdSe, Cu₂Se, Ag₂Se; organic selenium compounds, such as dimethylselenide, selenocystine, selenomethionine; selenate; and selenite. Selenate compounds are compounds of selenium with an oxidation number of 6, and include sodium selenate (Na₂SeO₄), and potassium selenate (K₂SeO₄). Selenite compounds are compounds of selenium with an oxidation number of 4, and include sodium selenite (Na₂SeO₃), and potassium selenite (K₂SeO₃). In particular, since sodium selenite in powder form is easily soluble in water, it can be suitably added to colostrum as an additive herein.

The above mixture preparation may contain immunoglobulins, or more preferably immunoglobulins G, which are exceptionally effective in protecting postnatal domestic mammals from infectious diseases and therefore are essential to postnatal domestic mammals. For example, postnatal calves require a blood concentration of IgG of 1,000 mg/dl or more; and, if not so satisfied, calves are vulnerable to infection of pathogenic microbes.

The above mixture preparations as feeds may contain extractions of immunoglobulins from colostrum, plasma-based ingredients, and/or cheese whey. In particular, the mixture preparations may contain raw milk, colostrum, and/or cheese whey, which has not gone through heat-sterilization processes. Colostrum, having gone through heat-sterilization processes, contains less immunoglobulins. Further, raw milk, colostrum, and/or cheese whey may be of a powdered form for easy storage and use. The mixture preparations are ordinarily administered to mammals by oral administration, etc.

Colostrum generally refers to milk secreted within one week post-parturition by maternal mammals. With respect to colostrum, the first milk secreted post-parturition is rich in immunoglobulins, and after the second milking, its immunoglobulin concentration rapidly decreases. Accordingly, it is preferable to use the first milking colostrum post-parturition. Moreover, since the colostrum secreted by a non-primiparous mother contains a higher concentration of immunoglobulins than that secreted by a primiparous mother, the former colostrum is preferable.

Another aspect of the present invention relates to a method of enhancing absorption of immunoglobulins in postnatal domestic mammals, which comprises administering a mixture preparation containing an addition of a selenium compound to a postnatal domestic mammal within 24 hours postpartum. After the addition of selenium to such mixture preparation, its concentration of selenium reaches 0.2 to 5 ppm, or more preferably 2.5 to 3.5 ppm. As the absorption capacity of immunoglobulins in postnatal domestic mammals decrease drastically postpartum as a function of time, the administration should be made within 8 hours postpartum, or preferably 3 hours postpartum.

The above mixture preparations are ordinarily administered to mammals by oral administration, etc. Of course, colostral feeding is available as well. In such cases, the one-time feeding of said mixture preparation of colostrum is provided at 0.3 to 3 liters, or preferably at 0.85 to 1.15 liters. The upper and lower limits are generally available from the Japanese Husbandry Standards, or the Nutrient Requirements of Dairy Cattle (7th rev. ed., pp. 221). In particular, the Japanese Husbandry Standards recommend feeding of colostrum of 1-2 liters within 4 hours postpartum, and 2 liters within 4-6 hours postpartum. Further, the Nutrient Requirements of Dairy Cattle recommend feeding of colostrum of at least 3 liters within one hour postpartum.

Another embodiment of the present invention is a method of enhancing absorption of immunoglobulins in postnatal domestic mammals, wherein said administration involves one-time feeding of said mixture preparation or multiple feedings of said mixture preparation in allotment.

Still another embodiment of the present invention is a method of enhancing absorption of immunoglobulins in postnatal domestic mammals, wherein said administration involves an one-time feeding of said mixture preparation within 3 hours postpartum and another one-time feeding of the same within 12 hours postpartum. As for the initial supplying of a mixture preparation of colostrum, it is necessary that such supply be made within 3 hours postpartum in order to enhance absorption of immunoglobulins in postnatal domestic mammals. As for the second-round of administration of a mixture preparation within 12 hours postpartum, it is carried out to supplement absorption of immunoglobulins therein.

### EXAMPLE

Hereinafter, the present invention will be described by using an example, but is not to be limited by such example.

In the example, colostrum added with selenium was administered to one of the two calves (Holstein types) in a pair, and colostrum without selenium was administered to the other calf. A total of 32 pairs of calves were employed for experimental purposes. For equalizing the experimental conditions of these two sets of calves, they were selected based on their proximities in body weight. The body weights ranged from 30.8-56.9 kg, and the differences between in body weight within a pair were less than 2.5 kg.

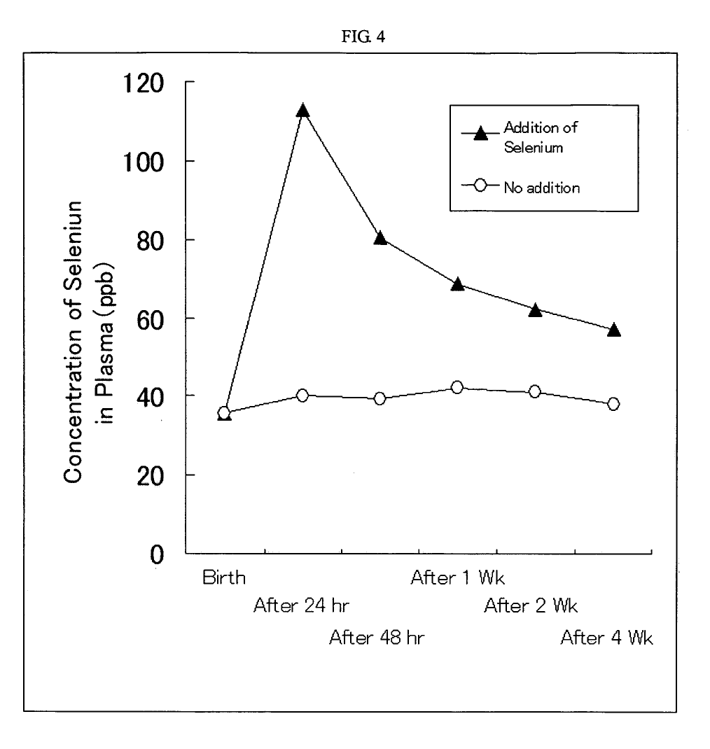

As a supply source of selenium, sodium selenite was used. In order to avoid different absorption rates of immunoglobulins based on different types of colostrum, the same amounts of colostrum milked from the same source were fed to these two calves in a pair. The colostrums fed (frozen for preservation) were of the four types as follows: the first milking post-parturition (Colostrum 1), the second milking post-parturition (Colostrum 2), the third milking post-parturition (Colostrum 3), and the fourth milking post-parturition (Colostrum 4). 2 liters of Colostrum 1 (1 L×2 bottles) were taken, and for the rest, 4 liters (2 L×2 bottles) were taken, respectively. 1 liter of Colostrum 1 within 2 hours post-parturition was fed, and then 2 liters of Colostrum 2, 3 and 4 were fed, respectively 12, 24 and 36 hours post-parturition. The concentrations of immunoglobulins in the colostrums used were 24.3-129.0 mg/ml, and those of selenium were 0.06-0.1 ppm (before adding selenium). The concentrations of immunoglobulins G (IgG) in plasma of calves 24 hours post-parturition were measured by means of radial immuno-diffusion (RID). The experiments as above were carried out while varying the selenium content based on the number of addition of selenium, i.e., (1) adding selenium only to Colostrum 1 (1.0-5.0 ppm), and (2) adding selenium to all four types of colostrum (0.2-4.0 ppm). As a reference, a graph showing the selenium concentrations in plasma after administration is provided in FIG. 4.

The results of the experiments are set forth in the tables as below:

TABLE 1

| Administration of Se (1×) | Body weight (kg) | IgG in Plasma (mg/ml) |
|---|---|---|
| Addition of 1 ppm of Se | 42.3 | 17 |
| No addition | 43 | 14.9 |
| (Average of 3 calves) | | |
| Addition of 2 ppm of Se | 39.2 | 16 |
| No addition | 40.1 | 15.8 |
| (Average of 3 calves) | | |
| Addition of 3 ppm of Se | 44.2 | 21.5 |
| No addition | 44.4 | 17.8 |
| (Average of 6 calves) | | |
| Addition of 4 ppm of Se | 42.2 | 17.4 |
| No addition | 41.9 | 14.9 |
| (Average of 4 calves) | | |
| Addition of 5 ppm of Se | 43.5 | 23 |
| No addition | 43 | 22.6 |
| (Average of 4 calves) | | |

Table 1 shows the body weights and the amounts of IgG in plasma in calves under experiment. Measurements are average values of calves within a test set, each set ranging from 3 to 6 pairs of calves. Selenium was added only once to the colostrum feed (1×).

TABLE 2

| Administration of Se (4×) | Body weight (kg) | IgG in Plasma (mg/ml) |
|---|---|---|
| Addition of 0.2 ppm of Se | 42.1 | 25.7 |
| No addition | 46.0 | 20.1 |
| (Average of 3 calves) | | |
| Addition of 1.0 ppm of Se | 43.6 | 32.9 |
| No addition | 43.5 | 30.0 |
| (Average of 5 calves) | | |
| Addition of 5.0 ppm of Se | 40.9 | 24.6 |
| No addition | 42.2 | 28.1 |
| (Average of 4 calves) | | |

Table 2 shows the data set for cases, in which colostrum feeds with or without additions of selenium were fed four times to calves under experiment.

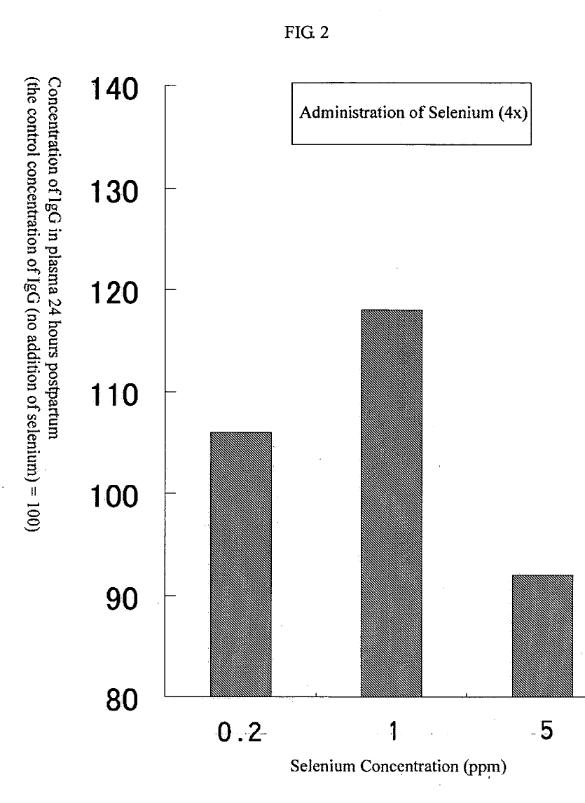

In FIGS. 1 and 2, the results of the experiments are summarized. The IgG concentration of calves administered with colostrum without selenium was set to be 100, and in such scale, the IgG concentrations of calves administered with colostrum with selenium are shown in FIGS. 1 and 2. With respect to one set of experimental conditions, 3 to 6 pairs of calves were involved. The average values therefrom are shown in FIGS. 1 and 2.

As shown in FIGS. 1 and 2, in most of the experimental conditions, the IgG concentrations in plasma of the calves administered with colostrum added with selenium were shown to be higher than those of the calves administered with colostrum without selenium. The IgG concentration in plasma of the calves fed four times with colostrum with selenium was about the same level as those of the calves administered with colostrum with selenium for the first feeding and the rest without selenium. Accordingly, the above result suggests that the addition of selenium to colostrum after the second feeding is not particularly effective in enhancing the absorption of immunoglobulins in calves but does serves as a supplementary boost.

Moreover, the blood samples were taken 24, 48 hours and 1 week post-parturition respectively, and biochemical blood tests (with 18 factors) were carried out to investigate any adverse effects of added selenium on the health conditions of calves. There was little difference between the test results of the calves administered with colostrum added with selenium, and those of the calves administered with colostrum without selenium. Accordingly, there seems to be no adverse effects of added selenium on the health conditions of calves.

Accordingly, the present invention enables enhancement of immunity strength of postnatal calves and reduction of their attrition rates, thereby resulting in breeding of healthy milk cows and cattle.

From the foregoing it will be appreciated that, although specific embodiments have been described herein for purposes of illustration, various modifications may be made without deviating from the spirit and scope of the invention. Accordingly, the invention is not limited except as by the appended claims and the elements recited therein.

1. A mixture preparation for enhancing absorption of immunoglobulins in postnatal domestic mammals, which comprises immunoglobulins and of a selenium compound in an amount that provides a concentration of 0.2 to 5 ppm of selenium.

2. The mixture preparation for enhancing absorption of immunoglobulins in postnatal domestic mammals according to claim 1, wherein said mixture preparation comprises 2.5 to 3.5 ppm of selenium.

3. The mixture preparation for enhancing absorption of immunoglobulins in postnatal domestic mammals according to claim 1, wherein said selenium compound is selected from the group consisting of sodium selenite, sodium selenate, potassium selenite, and potassium selenate.

4. (canceled)

5. The mixture preparation for enhancing absorption of immunoglobulins in postnatal domestic mammals according to claim 1, wherein said mixture preparation further comprises raw milk, colostrum or cheese whey.

6. A method of enhancing absorption of immunoglobulins in postnatal domestic mammals, which comprises administering to a postnatal domestic mammal within 24 hours postpartum a mixture preparation comprising a selenium compound in an amount that provides a concentration of 0.2 to 5 ppm of selenium.

7. The method of enhancing absorption of immunoglobulins in postnatal domestic mammals according to claim 6, wherein said mixture preparation contains 2.5 to 3.5 ppm of selenium.

8. The method of enhancing absorption of immunoglobulins in postnatal domestic mammals according to claim 6, wherein said selenium compound is of one or more of sodium selenite, sodium selenate, potassium selenite, and potassium selenate.

9. The method of enhancing absorption of immunoglobulins in postnatal domestic mammals according to claim 6, wherein said mixture preparation further comprises immunoglobulins.

10. The method of enhancing absorption of immunoglobulins in postnatal domestic mammals according to claim 6, wherein said mixture preparation further comprises raw milk, colostrum or cheese whey.

11. The method of enhancing absorption of immunoglobulins in postnatal domestic mammals according to claim 10, wherein said administration involves one-time feeding of said mixture preparation.

12. The method of enhancing absorption of immunoglobulins in postnatal domestic mammals according to claim 11, wherein said administration involves an one-time feeding of said mixture preparation within 3 hours postpartum and another one-time feeding of the same within 12 hours postpartum.

13. The method of enhancing absorption of immunoglobulins in postnatal domestic mammal according to claims 6, wherein said domestic mammal is a bovine.

14. The method of enhancing absorption of immunoglobulins in postnatal domestic mammals according to claim 10, wherein said administration involves multiple feedings of said mixture preparation in allotment.
